(19) United States
(12) Patent Application Publication
(10) Pub. No.: US 2023/0079025 A1
(43) Pub. Date: Mar. 16, 2023

(54) INFORMATION PROCESSING APPARATUS, INFORMATION PROCESSING METHOD, AND NON-TRANSITORY COMPUTER READABLE MEDIUM

(71) Applicants: CANON KABUSHIKI KAISHA, Tokyo (JP); CANON MEDICAL SYSTEMS CORPORATION, Tokyo (JP)

(72) Inventors: Toru Tanaka, Chiba (JP); Ryo Ishikawa, Kanagawa (JP); Tatsuya Kimoto, Tochigi (JP)

(21) Appl. No.: 17/939,217

(22) Filed: Sep. 7, 2022

(30) Foreign Application Priority Data
Sep. 10, 2021 (JP) 2021-148065
Sep. 10, 2021 (JP) 2021-148108

(51) Int. Cl. A61B 5/113; G06T 5/50; G06T 7/00; G06T 7/11; G06T 7/20; G06T 11/00; G06V 10/74

(52) U.S. Cl. CPC A61B 5/113; G06T 5/50; G06T 7/0012; G06T 7/11; G06T 7/20; G06T 11/003; G06V 10/761; G06T 2207/10116; G06T 2207/20221

(57) ABSTRACT

An information processing apparatus acquires projection data obtained by dividing a subject into first and second divided areas and capturing the first and second divided areas, the projection data including first projection data obtained by capturing a dynamic state of the subject in a first capturing range including the first divided area and second projection data obtained by capturing the dynamic state of the subject in a second capturing range including the second divided area. The apparatus acquires similarity relating to the dynamic state of the subject on a basis of projection data of a first partial area and projection data of a second partial area, and acquires a first timing for reconstructing an image of the first divided area and a second timing for reconstructing an image of the second divided area, on a basis of the similarity.

### BACKGROUND OF THE INVENTION

### Field of the Invention

The present disclosure relates to an information processing apparatus, an information processing method, and a non-transitory computer readable medium.

### Description of the Related Art

In a medical field, doctors make diagnoses using medical images captured with various modalities. Particularly, when sites, where disease conditions are more symptomatic in the movement of an internal organ such as a lung and a heart, are the targets for diagnoses, the diagnoses are sometimes made by using moving images composed of a plurality of medical images captured consecutively (that is, in a time series). As a modality able to capture three-dimensional tomographic images as moving images (in a time series), an area dictator type X-ray CT apparatus is available. The modality captures three-dimensional CT images as moving images and is therefore also called a 4DCT apparatus to which a time axis is added. Further, images captured by the 4DCT apparatus are also called 4DCT images.

The 4DCT apparatus has limitations on an image-capturing range when implementing single capturing. Therefore, in order to observe an internal organ larger in size than the capturing range, there is a case where capturing is performed a plurality of times in different capturing ranges and resulting images are combined together. For example, an attempt has been made in which 4DCT images of an upper part and a lower part of a lung are separately captured, and three-dimensional images, which have respiration phases corresponding to each other, are aligned and bonded together to generate a 4DCT image of the entire lung.

Further, Japanese Patent Application Laid-open No. 2020-141841 discloses a technology to analyze the dynamic state of a lung field in each of a plurality of moving images capturing the lung field and select frames having respiration phases corresponding to each other.

Generally, higher frame rates make it possible to match respiration phases to each other more precisely between a plurality of moving images. Japanese Patent Application Laid-open No. 2020-141841 discloses a technology of compensation of generating images between frames by interpolation when the number of the frames of one moving image is smaller than that of the other moving image.

However, when the phases of the dynamic state of an observation region are made to correspond to each other between a plurality of moving images in which an entire capturing range is reconstructed at a high frame rate, a problem in that a calculation amount becomes enormous.

### SUMMARY OF THE INVENTION

Therefore, the present disclosure has been made in view of the above problem and has an object of providing a technology to match the phases of the dynamic state of an observation region to each other with high accuracy between a plurality of moving images while suppressing a calculation amount required for reconstruction.

According to an aspect of the present disclosure, it is provided an information processing apparatus including at least one memory storing a program, and at least one processor which, by executing the program, causes the information processing apparatus to acquire projection data obtained by dividing a subject into a first divided area and a second divided area and capturing the first divided area and the second divided area, the projection data including first projection data obtained by capturing a dynamic state of the subject in a first capturing range including the first divided area and second projection data obtained by capturing the dynamic state of the subject in a second capturing range including the second divided area, acquire similarity relating to the dynamic state of the subject between the first projection data and the second projection data on a basis of projection data of a first partial area that is at least a part of the first capturing range in the first projection data and projection data of a second partial area that is at least a part of the second capturing range in the second projection data, and acquire a first timing for reconstructing an image of the first divided area from the first projection data and a second timing for reconstructing an image of the second divided area from the second projection data, on a basis of the similarity. In addition, according to an aspect of the present disclosure, it is provided an information processing apparatus including at least one memory storing a program, and at least one processor which, by executing the program, causes the information processing apparatus to acquire projection data obtained by dividing a subject into a first divided area and a second divided area and capturing the first divided area and the second divided area, the projection data including first projection data. obtained by capturing a dynamic state of the subject in a first capturing range including the first divided area and second projection data obtained by capturing the dynamic state of the subject in a second capturing range including the second divided area, acquire a moving image of a first partial area obtained by reconstructing an image of a first partial area that is a part of the first capturing range from the first projection data and a moving image of a second partial area obtained by reconstructing an image of the second partial area that is a part of the second capturing range from the second projection data, acquire similarity in the dynamic state of the subject between respective frames of the moving image of the first partial area and the moving image of the second partial area, and acquire a first timing for reconstructing an image of the first divided area from the first projection data and a second timing for reconstructing an image of the second divided area from the second projection data, on a basis of the similarity.

The present disclosure may be regarded as an image processing method that includes at least a part of the above mentioned processing, a program that causes a computer to execute this method, or a non-transitory computer readable recording medium that stores this program. The present invention may be implemented by combining the above mentioned configurations and processing operations as long as there is no technical inconsistency.

Further features of the present invention will become apparent from the following description of exemplary embodiments with reference to the attached drawings.

### BRIEF DESCRIPTION OF THE DRAWINGS

FIG. 1 is a diagram showing the entire configuration of an X-ray CT apparatus according to a first embodiment;

FIG. 2 is a diagram showing the equipment configuration of an information processing apparatus in the first embodiment;

FIG. 3 is a flowchart showing an example of an entire processing procedure in the first embodiment;

FIG. 4 is a diagram showing an example of capturing ranges in the first embodiment;

FIGS. 5A and 5B are diagrams showing an example of acquiring a reconstruction timing in the first embodiment;

FIG. 6 is a diagram showing the equipment configuration of an information processing apparatus in a second embodiment;

FIG. 7 is a flowchart showing an example of an entire processing procedure in the second embodiment; and

FIG. 8 is a diagram showing an example of a sinogram in the second embodiment.

### DESCRIPTION OF THE EMBODIMENTS

Hereinafter, embodiments of the present disclosure will be described with reference to the drawings. Note that the present disclosure is not limited to the following embodiments but is appropriately modifiable without departing from its scope. Further, units having the same functions are denoted by the same symbols, and their descriptions may be omitted or simplified in the following drawings.

### First Embodiment

An information processing apparatus according to the present embodiment is an apparatus that generates, from three-dimensional moving images obtained by dividing an observation region showing a cyclic dynamic state and capturing the same a plurality of times, a combined moving image in which respective frame images are combined together with the phases of the dynamic state of the observation region matched to each other between the plurality of moving images.

The information processing apparatus of the present embodiment divides an area into two areas (a first divided area and a second divided area) so that at least parts of the areas of an observation region are overlapped with each other and sets a first capturing range and a second capturing range so that the respective divided areas are captured. Then, the information processing apparatus captures the dynamic state of the observation region with an X-ray CT apparatus in a time series using the first capturing range and the second capturing range to acquire first projection data and second projection data.

Next, the information processing apparatus of the present embodiment reconstructs a plurality of (time-series) images from the respective projection data at prescribed frame rates for areas (partial areas) having overlapped capturing ranges between the first projection data and the second projection data to acquire moving images (three-dimensional moving images). That is, the information processing apparatus acquires, from the first projection data, a moving image of a first partial area reconstructed for the first partial area having a capturing range overlapped with the capturing range of the second projection data. Similarly, the information processing apparatus acquires, from the second projection data, a moving image of a second partial area reconstructed for the second partial area having a capturing range overlapped with the capturing range of the first projection data.

At this time, the respective frame rates (time resolution) of the moving image of the first partial area and the moving image of the second partial area are desirably higher than the frame rate (time resolution) of a combined moving image that is generated in subsequent processing. In the subsequent processing, the information processing apparatus acquires a timing at which the phases of dynamic state of the observation region are matched to each other between the moving image of the first partial area and the moving image of the second partial area, and reconstructs frame images used to generate a combined moving image at the acquired timing. When the frame rates of the moving image of the first partial area and the moving image of the second partial area are lower than that of the combined moving image, the phases of the dynamic state of the observation region are coarser than that at the frame rate of the combined moving image. Therefore, it is difficult to acquire a reconstruction timing (the phases of the dynamic state) suitable for the respective frame images of the combined moving image. On the other hand, when the frame rates of the moving image of the first partial area and the moving image of the second partial area are higher than that of the combined moving image, the phases of the dynamic state of the observation region are denser than that at the frame rate of the combined moving image. Therefore, it is possible to acquire a reconstruction tinning (the phases of the dynamic state) more suitable for the respective frame images of the combined moving image.

Next, the information processing apparatus of the present embodiment acquires the similarity of the observation region between the respective frames of the moving image of the first partial area and the moving image of the second partial area. On the basis of the similarity of the observation region, the information processing apparatus acquires, from the first projection data, a first timing for reconstructing the first projection data in the entire first capturing range (that is, the first divided area) (a plurality of time positions at which reconstruction processing is performed). Further, the information processing apparatus acquires, from the second projection data, a second timing for reconstructing the second projection data in the entire second capturing range (that is, the second divided area) (a plurality of time positions at which reconstruction processing is performed).

In addition, the information processing apparatus of the present embodiment acquires a moving image of the first divided area obtained by reconstructing an image from the first projection data at the first timing and a moving image of the second divided area obtained by reconstructing an image from the second projection data at the second timing. Then, the information processing apparatus generates a combined moving image in which the moving image of the first divided area and the moving image of the second divided area are combined together so that the observation regions are matched to each other. That is, in the present embodiment, on the basis of a comparison between moving images obtained by reconstructing partial areas having overlapped capturing ranges, the information processing apparatus determines timings necessary for generating a combined moving image for respective projection data, and reconstructs moving images of respective divided areas for the projection data at the timings. Thus, the information processing apparatus is allowed to limit a timing at which the reconstruction of the entire capturing range is necessary and reduce a calculation amount for the reconstruction. Further, by reconstructing only moving images of partial areas at high frame rates, the information processing apparatus is allowed to make first projection data and second projection data correspond to each other with high time resolution while reducing a calculation amount for reconstruction.

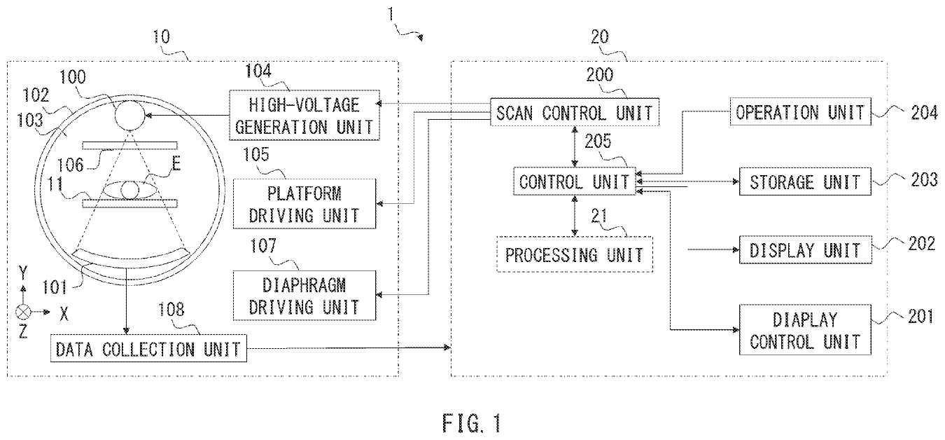

Hereinafter, the configuration and processing of the present embodiment will be described using FIG. 1 to FIGS. 5A and 5B. Note that a description will be given using a three-dimensional moving image obtained by capturing the respiratory movement of a lung as an example in the present embodiment. However, the present embodiment is not limited to this but is applicable also to a moving image obtained by capturing an arbitrary region such as a heart that performs voluntary movement, a moving image obtained by capturing an arbitrary region for which a subject has performed cyclic movement (for example, a bending exercise or the like.

FIG. 1 shows the entire configuration of an X-ray CT apparatus according to the present embodiment. As shown in FIG. 1, an X-ray CT apparatus 1 includes a platform apparatus 10, a bed apparatus 11, and a console apparatus 20.

The platform apparatus 10 is an apparatus that irradiates a subject E with X-rays and collects scan data (detection data) of the X-rays having passed through the subject E. The platform apparatus 10 has an X-ray generation unit 100, an X-ray detection unit 101, a rotation body 102, a high-voltage generation unit 104, a platform driving unit 105, an X-ray diaphragm unit 106, a diaphragm driving unit 107, and a data collection unit 108.

The X-ray generation unit 100 includes an X-ray tube bulb (for example, a vacuum tube (not shown) that generates conical or pyramidal X-ray beams). The X-ray generation unit 100 irradiates the subject E with the generated X-rays.

The X-ray detection unit 101 includes a plurality of X-ray detection elements (not shown). The X-ray detection unit 101 detects X-rays having passed through the subject E. Specifically, the X-ray detection unit 101 detects X-ray intensity distribution data showing the intensity distribution of X-rays having passed through the subject E with the X-ray detection elements and generates the detection data as an electric signal. After amplifying the generated electric signal, the X-ray detection unit 101 converts the electric signal into a digital signal and outputs the same. As the X-ray detection unit 101, a two-dimensional X-ray detector (surface detector) in which a plurality of detection elements are arranged in both two directions (a slice direction and a channel direction) orthogonal to each other is, for example, used. The plurality of X-ray detection elements are provided in, for example, 320 lines along the slice direction. Through the use of the plurality of X-ray detectors provided with X-ray detection elements in multiple lines as described above, it is possible to capture a three-dimensional capturing area having a width in the slice direction with a one-rotation scan (volume scan). Note that the slice direction corresponds to the body axis direction of the subject E, and the channel direction corresponds to the rotation direction of the X-ray generation unit 100.

The rotation body 102 is a member that supports the X-ray generation unit 100 and the X-ray detection unit 101 so as to face each other with the subject E interposed therebetween. The rotation body 102 has an opening 103 penetrating in the slice direction. Inside the platform apparatus 10, the rotation body 102 is arranged so as to rotate in a circular orbit about the subject E. That is, the X-ray generation unit 100 and the X-ray detection unit 101 are rotatably provided along the circular orbit about the subject E.

The high-voltage generation unit 104 applies a high voltage to the X-ray generation unit 100 (hereinafter, a “voltage” represents a voltage between an anode and a cathode in an X-ray tube bulb). The X-ray generation unit 100 generates X-rays on the basis of the high voltage.

The platform driving unit 105 rotationally drives the rotation body 102. The X-ray diaphragm unit 106 has a slit (opening) with a prescribed width and changes the width of the slit to adjust a fan angle (a spread angle in the channel direction) and a cone angle (a spread angle in the slice direction) of X rays irradiated from the X-ray generation unit 100. The diaphragm driving unit 107 drives the X-ray diaphragm unit 106 so that X rays generated by the X-ray generation unit 100 are formed into a prescribed shape.

The data collection unit 108 (DAS: Data Acquisition System) collects detection data from the X-ray detection unit 101 (respective X-ray detection elements). Then, the data collection unit 108 transmits the detection data that is a digital signal to the console apparatus 20.

The bed apparatus 11 is an apparatus on which the subject E to be captured is mounted and moved. The bed apparatus 11 is able to move in the body axis direction of the subject E and a direction orthogonal to the body axis direction. That is, the bed apparatus 11 is able to insert and remove a bed on which the subject E is mounted into and from the opening 103 of the rotation body 102.

In the present embodiment, the bed apparatus 11 is inserted into and removed from the opening 103 of the rotation body 102 so that at least parts of the areas (capturing ranges) of an observation region of the subject E are overlapped with each other, and the X-ray generation unit 100 irradiates the subject E with X-rays at respective capturing positions. Thus, first detection data and second detection data of which at least parts of the areas of an observation region are overlapped with each other are collected. The first detection data and the second detection data are time-series data collected by continuously performing a scan (dynamic scan) for a prescribe period while the observation region of the subject E performs movement.

The console apparatus 20 is used to perform an operation input to the X-ray CT apparatus. Further, the console apparatus 20 has the function of reconstructing CT image data (tomographic image data or volume data) showing an inner form of the subject E from detection data collected by the platform apparatus 10, or the like. The console apparatus 20 includes a processing unit 21, a scan control unit 200, a display control unit 201, a display unit 202, a storage unit 203, an operation unit 204, and a control unit 205.

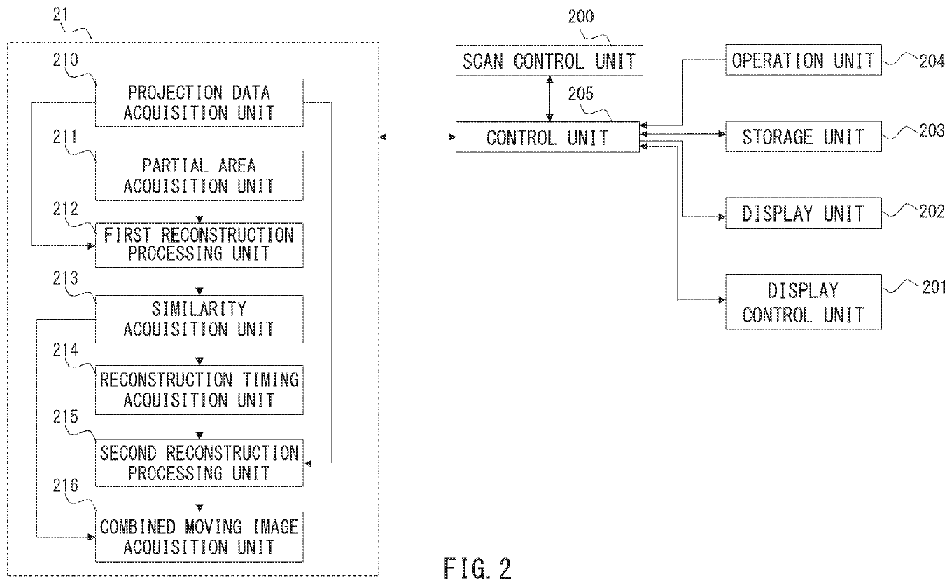

FIG. 2 shows the configuration of the information processing apparatus (processing unit 21) according to the present embodiment together with the control unit or the like shown in FIG. 1. In FIG. 2, the same units as those shown in FIG. 1 are denoted by the same symbols, and their descriptions will be omitted appropriately.

The processing unit 21 performs various processing on detection data (first detection data and second detection data) transmitted from the platform apparatus 10 (data collection unit 108). The processing unit 21 includes a projection data acquisition unit 210, a partial area acquisition unit 211, a first reconstruction processing unit 212, a similarity acquisition unit 213, a reconstruction timing acquisition unit 214, a second reconstruction processing unit 215, and a combined moving-image acquisition unit 216.

The projection data acquisition unit 210 performs processing such as logarithmic conversion processing, offset correction, sensitivity correction, and beam hardening correction on each of first detection data and second detection data detected by the platform apparatus 10 (X-ray detection unit 101). Thus, the projection data acquisition unit 210 generates first projection data and second projection data. That is, projection data is obtained by capturing the subject E with the platform apparatus 10.

The partial area acquisition unit 211 acquires, on the basis of capturing positions controlled by the scan control unit 200, a first partial area in which the first reconstruction processing unit 212 performs reconstruction processing on first projection data and a second partial area in which the first reconstruction processing unit 212 performs reconstruction processing on second projection data.

The first reconstruction processing unit 212 performs reconstruction processing on first projection data generated by the projection data acquisition unit 210 in a first partial area acquired by the partial area acquisition unit 211 to generate a moving image (4DCT image) of the first partial area. Further, the first reconstruction processing unit 212 performs reconstruction processing on second projection data generated by the projection data acquisition unit 210 in a second partial area acquired by the partial area acquisition unit 211 to generate a moving image of the second partial area.

It is possible to change a reconstruction condition. For the reconstruction of a tomographic image, an arbitrary method such as a two-dimensional Fourier transform method and a convolution back projection method is, for example, available. Volume data is generated by performing interpolation processing on a plurality of reconstructed tomographic image data. For the reconstruction of volume data (3DCT image), an arbitrary method such as a cone beam reconstruction method, a multi-slice reconstruction method, and an enlargement reconstruction method is, for example, available. By performing a volume scan with an X-ray detector in which a plurality of X-ray detection elements are provided in multiple lines as described above, it is possible to reconstruct wide-range volume data.

The similarity acquisition unit 213 compares a moving image of a first partial area and a moving image of a second partial area that are acquired by the first reconstruction processing unit 212 with each other to acquire the similarity of an observation region between the moving image of the first partial area and the moving image of the second partial area. The similarity of the observation region is information that specifies, from respective frame images of the moving image of the first partial area (frame images of the first partial area), frame images of the moving image of the second partial area (frame images of the second partial area) having a close phase within respective cycles of a cyclically-moving observation region.

The reconstruction timing acquisition unit 214 sets, on the basis of the similarity of an observation region acquired by the similarity acquisition unit 213, a timing (first timing) for reconstructing first projection data in an entire capturing range (that is, a first divided area). Further, the reconstruction timing acquisition unit 214 sets, on the basis of the similarity of the observation region, a timing (second timing) for reconstructing second projection data in an entire capturing range (that is, a second divided area).

The second reconstruction processing unit 215 acquires a moving image of a first divided area obtained by reconstructing first projection data at a timing acquired by the reconstruction timing acquisition unit 214 and a moving image of a second divided area obtained by reconstructing second projection data at a timing acquired by the reconstruction timing acquisition unit 214.

The combined moving-image acquisition unit 216 acquires, on the basis of the similarity of an observation region acquired by the similarity acquisition unit 213, a combined moving image in which respective frame images of a moving image of a first divided area and a moving image of a second divided area are combined together.

The scan control unit 200 controls various operations relating to an X-ray scan. For example, the scan control unit 200 controls the high-voltage generation unit 104 to apply a high voltage to the X-ray generation unit 100. The scan control unit 200 controls the platform driving unit 105 to rotationally drive (rotates and drives) the rotation body 102. The scan control unit 200 controls the diaphragm driving unit 107 to operate the X-ray diaphragm unit 106. The scan control unit 200 controls the movement of the bed apparatus 11.

The display control unit 201 performs various control relating to an image display. For example, the display control unit 201 performs control to cause the display unit 202 to display a combined moving image acquired by the combined moving-image acquisition unit 216.

The display unit 202 is constituted by an arbitrary display device such as a LCD (Liquid Crystal Display) and a CRT (Cathode Ray Tube) display. For example, a MPR (Multi Planer Reconstruction) image obtained by performing rendering processing on respective frame images of a combined moving image is reproduced and displayed on the display unit 202 in time series.

The storage unit 203 is constituted by a storage unit such as a RAM (Random Access Memory) and a ROM (Read Only Memory). The storage unit 203 stores detection data, projection data, a 4DCT image and a combined moving image after having been applied to reconstruction processing, or the like.

The operation unit 204 is used as an input device to perform various operations on the console apparatus 20. The operation unit 204 is constituted by, for example, a keyboard, a mouse, a trackball, a joystick, or the like. Further, it is also possible to use a GUI (Graphical User Interface), displayed on the display unit 202, as the operation unit 204. In the present embodiment, the operation unit 204 is used to set a reconstruction condition or change a display state of a generated image (for example, the position of a cross section, the direction of a cross section, or the like in a MPR image or the magnification of an image).

The control unit 205 controls the operations of the platform apparatus 10, the bed apparatus 11, and the console apparatus 20 to perform the entire control of the X-ray CT apparatus. For example, the control unit 205 controls the scan control unit 200 to cause the same to scan the platform apparatus 10 and collect detection data. Further, the control unit 205 controls the processing unit 21 to cause the same to perform various processing (acquisition of projection data, reconstruction processing, and acquisition of a combined moving image) on detection data. Alternatively, the control unit 205 controls the display control unit 201 to cause the same to display a combined moving image or the like on the display unit 202 on the basis of image data or the like stored in the storage unit 203.

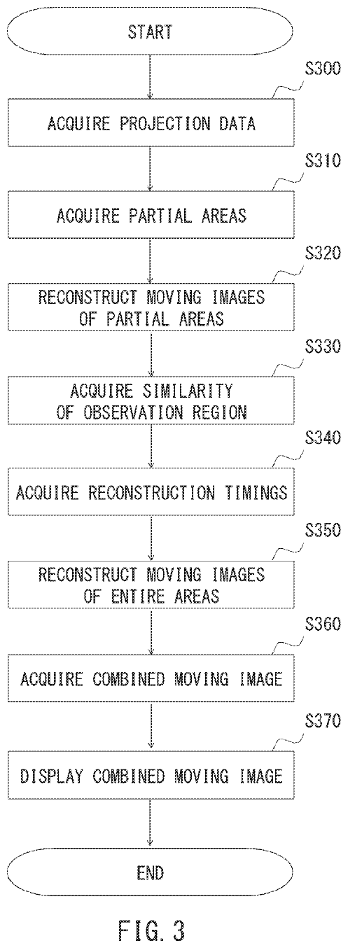

FIG. 3 shows the flowchart of an entire processing procedure performed by the information processing apparatus (console apparatus 20) according to the present embodiment. In the following description, a lung will be assumed as an observation region.

<S300: Acquisition of Projection Data> In step S300, the projection data acquisition unit 210 generates first projection. data and second projection data from first detection data and second detection data that are collected by the platform apparatus 10, respectively, and acquires the same. Then, the projection data acquisition unit 210 outputs both the acquired first projection data and second projection data to the first reconstruction processing unit 212 and the second reconstruction processing unit 215.

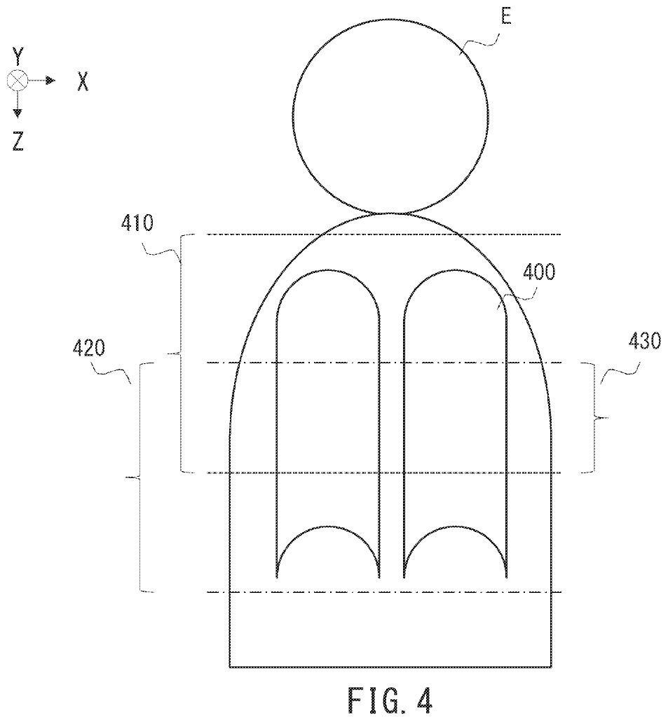

In the present embodiment, the first projection data and the second projection data are projection data obtained by dividing the lung in its head-tail direction so that at least parts of the areas of the lung are overlapped with each other and capturing the same. Further, the capturing range (that is, a first divided area) of the first projection data is an upper part of the lung that includes the apex of the lung, and the capturing range (that is, a second divided area) of the second projection data is a lower part of the lung that includes the base of the lung. FIG. 4 schematically shows an example of capturing ranges. When a lung 400 of a subject E is an observation region in FIG. 4, projection data obtained by capturing a range 410 is the first projection data and projection data obtained by capturing a range 420 is the second projection data. A range 430 is an area in which the parts of the lung are overlapped with each other.

The projection data of the present embodiment takes data generated from detection data collected by an X-ray CT apparatus as an example and is data including a sinogram. The sinogram draws the decay of X-rays as the function of a “space” (horizontal axis) along an X-ray detector (X-ray detection unit 101) and an “angle” (vertical axis) of a scan in measurement performed at a series of projection angles. The dimension of the space is a position along the one-dimensional arrangement of the X-ray detector. Further, the dimension of the angle is an X-ray projection angle that changes as the function of time, which represents that a projection angle increases in equal amounts with time and projection measurement is performed at a series of linearly-changing projection angles. The decay of X-rays resulting from a certain specific volume (for example, a lung) in a body draws a sine wave about a vertical axis. However, the amplitude of the sine wave becomes larger as the volume is distant from the center of rotation, and the phase of the sine wave fixes the angular position of the volume with respect to the axis of the rotation. In the present embodiment, an inverse Radon transform or an equivalent image reconstruction method is performed, whereby an image is reconstructed from projection data in a sinogram. The reconstructed image corresponds to one tomographic image data of a body.

<S310: Acquisition of Partial Areas> In step S310, the partial area acquisition unit 211 acquires, on the basis of capturing positions controlled by the scan control unit 200, a first partial area in which the first reconstruction processing unit 212 performs reconstruction processing on the first projection data. Similarly, the partial area acquisition unit 211 acquires a second partial area in which the first reconstruction processing unit 212 performs reconstruction processing on the second projection data. Then, the partial area acquisition unit 211 outputs the acquired first and second partial areas to the first reconstruction processing unit 212.

In the present embodiment, the partial area acquisition unit 211 regards an area (the range 430 shown in FIG. 4), in which the capturing ranges of the first detection data and the second detection data that are fixed on the basis of the capturing positions controlled by the scan control unit 200 are overlapped with each other, as a partial area. Note that the first partial area and the second partial area may be areas entirely including the area in which the capturing ranges are overlapped with each other, or may be areas partially including the area in which the capturing ranges are overlapped with each other. Further, the first partial area and the second partial area may not necessarily include the area in which the capturing ranges are overlapped with each other.

Further, the first partial area and the second partial area in which the reconstruction processing is performed may be areas same or different from each other in a real space. When the first partial area and the second partial area are the areas same in the real space, both the first partial area and the second partial area may correspond to, for example, the range 430 shown in FIG. 4 or a partial area included in the range 430. When the first partial area and the second partial area are areas different from each other in the real space, one of the first partial area and the second partial area may correspond to, for example, the range 430 and the other thereof may correspond to, for example, a partial area included in the range 430.

Further, the partial area may be an area in which the capturing ranges are not overlapped with each other. The first partial area may be any part of the range 410 including at least a part of the range 430, or may be the entirety of the range 410. Similarly, the second partial area may be any part of the range 420 including at least a part of the range 430, or the first partial area may be the entirety of the range 410. Alternatively, the first partial area may be a part of the range 410 excluding the range 430, and the second partial area may be a part of the range 420 excluding the range 430. Thus, it is possible to make the projection data of the first partial area and the projection data of the second partial area, each of which shows a different area in an observation region, correspond to each other with high time resolution.

<S320: Acquisition of Moving Images of Partial Areas> In step S320, the first reconstruction processing unit 212 performs reconstruction processing on the first projection data acquired in step S300 in the first partial area acquired in step S310 to acquire a moving image of the first partial area. Further, the first reconstruction processing unit 212 performs reconstruction processing on the second projection data acquired in step S300 in the second partial area acquired in step S310 to acquire a moving image of the second partial area. Then, the first reconstruction processing unit 212 outputs the acquired moving images of the first and second partial areas to the similarity acquisition unit 213.

Here, a reconstruction frame rate in the present step is desirably set at the value of high time resolution to such an extent that an image having the substantially same phase exists between the moving image of the first partial area and the moving image of the second partial area. In the present embodiment, the reconstruction of the moving images of the first and second partial areas is performed at a prescribed frame rate (for example, 10 frames/second). The frame rate may be the same or different between the moving images of the first and second partial areas. The frame rate may be set by a user via the operation unit 204. For example, the frame rate suitable for an observation region may be set by the user via the operation unit 204. Alternatively, the frame rate may be one obtained by equally dividing respective time lengths, at which the first projection data and the second projection data are acquired, by a prescribed frame number.

Further, the first reconstruction processing unit 212 may automatically set the frame rate according to the name of an observation region. At this time, the name of the observation region may be specified by the user, or may be automatically determined according to capturing parameters used to capture the observation region by the platform apparatus 10. For example, the time interval of one cycle is different in cyclic movement between the heart rate of a heart and the respiration of a lung. Accordingly, when the name of the observation region is a heart (when information indicating that the name of the observation region is a heart is acquired), the frame rate may be set to be high. If the heart is included in a capturing range at divided capturing even when the name of the observation region is a lung, the frame rate may be set to be higher to suit the movement cycle of the heart. That is, as for an upper part of the lung (the moving image of the first partial area) in which only the lung moves inside a capturing range, the reconstruction is performed at the frame rate previously set for the lung. In addition, as for a lower part of the lung (the moving image of the second partial area) in which the heat is also included in a capturing range together with the lung, the reconstruction is performed at the frame rate previously set for the heart. Thus, it is possible to acquire a moving image picking up a state in which the lung deforms due to the influence of the heart rate of the heart at capturing the lower part of the lung.

Further, the frame rate for the reconstruction may not be constant in one moving image. For example, the frame rate for a time range in which the movement of the observation region is slow may be set to be low, and the frame rate for a time range in which the movement is fast may be set to be high to perform the reconstruction. The change speed of the dynamic state of the observation region may be acquired through the analysis of projection data, or may be acquired through the observation of a heart rate or a respiration cycle with an external apparatus not shown. As a method for acquiring the change speed of the dynamic state of the observation region through the analysis of projection data, a known method is available. For example, when there is a large difference between opposite data of projection data collected by the X-ray CT apparatus 1 (two sinograms having the same “space” and “times (angles)” different by 180 degrees), it is possible to determine that the movement of the observation region is fast at the time at which the opposite data has collected.

<S330: Acquisition of Similarity of Observation Region> In step S330, the similarity acquisition unit 213 acquires the similarity of the observation region between respective frame images of the first partial area and respective frame images of the second partial area that are acquired in step S320. An example of the similarity of the observation region is image similarity between the frame images, and the similarity acquisition unit 213 acquires the image similarity between the respective frame images of the first partial area and the respective frame images of the second partial area. In the present embodiment, the similarity acquisition unit 213 acquires, in order to acquire more appropriate similarity, image similarity obtained when relative positions between the frame images are translated at a plurality of patterns. Then, the similarity acquisition unit 213 acquires a translational movement amount at which the image similarity becomes the highest and the image similarity as the similarity of the observation region. Then, the similarity acquisition unit 213 outputs the similarity of the observation region acquired in the above processing to the reconstruction timing acquisition unit 214 and the combined moving-image acquisition unit 216.

The present embodiment will specifically describe a method for acquiring the similarity of an observation region, taking a case in which the observation region is a lung and the similarity of the observation region is image similarity as an example. The image similarity is calculated between prescribed frame images of a first partial area and prescribed frame images of a second partial area. At this time, due to positional deviations resulting from the body movement of a subject, there is a possibility that a prescribed region originally drawn at the same position between the prescribed frame images of the first partial area and the prescribed frame images of the second partial area is drawn at different positions between the respective images. In order to address this problem, the similarity acquisition unit 213 moves and corrects the positions of the frame images of the second partial area relative to the positions of the frame images of the first partial area to calculate the image similarity.

When the lung is an observation region, body movement in a body axis direction becomes an issue due to respiration. Therefore, the similarity acquisition unit 213 translates the positions of the frame images of the second partial area in the body axis direction to calculate the image similarity. At this time, the similarity acquisition unit 213 calculates the respective image similarity for each of a plurality of translational movement amounts at which the images are translated, and acquires a translational movement amount at which the image similarity becomes the highest and the image similarity. Further, the similarity acquisition unit 213 may not necessarily correct the positions with a plurality of translational movement amounts. For example, the similarity acquisition unit 213 may correct the positions of the respective frame images of the second partial area with a single translational movement amount and calculate the image similarity between the respective frame images of the first partial area and the respective frame images of the second partial area. At this time, the single translational movement amount may be a value specified by a user in advance, or may be a difference between the position of the first partial area and the position of the second partial area (for example, a central position in the body axis direction). Further, the similarity acquisition unit 213 may calculate the image similarity without correcting the positions (translational movement amount=0).

As the image similarity, a known method such as Sum of Squared Difference (SSD), a mutual information amount, and a mutual correlation coefficient is available. When the image similarity is calculated, areas of interest may be set. For example, a lung field may be extracted from the respective prescribed frame images of the first and second partial areas, and the image similarity may be calculated only for the lung field. As processing to extract the lung field, a known image processing method is available. Arbitrary threshold processing using the threshold of a pixel value or known segmentation processing including graph cut processing may be used. Alternatively, the image similarity may be calculated for an image haying been subjected to preprocessing such as pixel value conversion under a condition suitable for the observation of the lung field (for example, a window level (WL) is −600, and a window width (WW) is 1500)

The similarity of the observation region may be acquired by a method other than the image similarity. For example, the proximity of a phase within a cycle of the observation region showing a cyclic dynamic state may be acquired as similarity. When the observation region is a lung, the respiration phase of the lung in respective frame images are estimated in each of a moving image of a first partial area and a moving image of a second partial area. Then, the proximity of a respiration phase within the respiration cycle of the respective frame images of the first partial area and the respective frame images of the second partial is acquired as similarity. According to this method, a phases is independently estimated for each of the moving image of the first partial area and the moving image of the second partial area. Therefore, it is possible to acquire the similarity of the observation region even when capturing ranges are not overlapped with each other.

The respiration phase of the lung may be calculated through the analysis of frame images. For example, an image feature amount that varies together with a respiration phase is available. As the image feature amount, the volume or area of a lung field, an average of the pixel values of a lung field, and the position of a trackable and characteristic region such as a body surface position is available. The similarity of the image feature amount may be regarded as the similarity of an observation region. That is, a difference between an image feature amount calculated from a prescribed frame image of a first partial area and an image feature amount calculated from a prescribed frame image of a second partial area may be regarded as similarity.

Further, the respiration phase may be acquired through the analysis of the movement of the lung in a moving image instead of an image feature amount. For example, the movement of the lung may be analyzed on the basis of the timewise change rate of the image feature amount, and the similarity of an observation region may be acquired on the basis of the analysis result of the movement. The fact that the image feature amount cyclically varies together with a respiration phase and correlates with the change speed of the dynamic state of the lung and the time vise change rate of the image feature amount is used. Specifically, the image feature amount is calculated in respective frame images of a first partial area, and a first curve (for example, a sine curve) is applied to the transition of the image feature amount. Similarly, the image feature amount is calculated in respective frame images of a second partial area, and a second curve is applied to the transition of the image feature amount. Further, the similarity of parameters (phases in the case of a sine curve) may be acquired as the similarity of the observation region between the first and second curves.

Note that the above example describes an example in which the positions are corrected by the translational movement. However, the positions may be corrected using rotation, enlargement, or contraction (affine transformation), besides the translational movement. Further, the positions may be corrected through the use of a nonlinear deformation model. As the deformation model, a deformation model based on a radial basis function such as Thin Plate Spline (TPS) or a known deformation model such as Free Form Deformation (FFD) is available.

Further, in the above example, the image similarity is calculated for each of the plurality of translational movement amounts, and the translational movement amount at which the image similarity becomes the highest and the image similarity are acquired. However, a combination of the respective translational movement amounts and the image similarity may be acquired as the similarity of the observation region.

<S340: Acquisition of Reconstruction Timings> In step S340, the reconstruction timing acquisition unit 214 acquires, on the basis of the similarity of the observation region acquired in step S330, time positions at which phases correspond to each other within the cycle of the observation region showing a cyclic dynamic state in time ranges in which the first projection data. and the second projection data have been captured. Then, the reconstruction timing acquisition unit 214 regards time positions acquired from a time range in which the first projection data has been captured as a first timing and time positions acquired from a time range in which the second projection data has been captured as a second timing, and outputs the timings to the second reconstruction processing unit 215.

In the present embodiment, a matching method according to Dynamic Programming (DP) is used as an example of acquiring an optimum combination of the first timing and the second timing with respect to the two input systems of the moving image of the first partial area and the moving image of the second partial area. In the DP matching of the present embodiment, a cost minimization problem assuming the similarity of the observation region between the frame images of the respective moving images acquired in step S330 as a cost is solved to acquire the optimum combination.

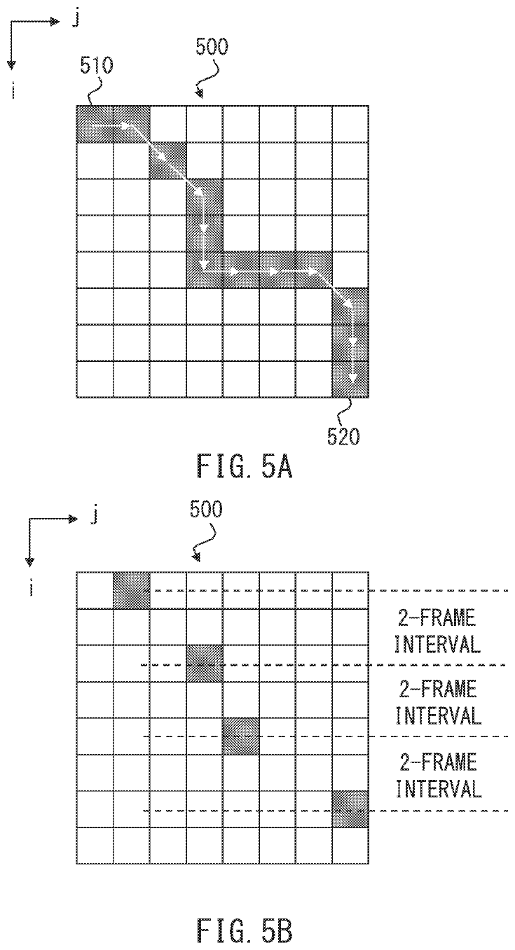

The acquisition will be specifically described using FIGS. 5A and 5B. A matrix 500 shown in FIG. 5A is a matrix storing the similarity of an observation region in a case in which i represents a frame number (i=1, 2, . . . , M) of a moving image of a first partial area and j represents a frame number (j=1, 2, . . . , N) of a moving image of a second partial area. For example, when the similarity of the observation region is image similarity, the image similarity between the first frame image of the first partial area and the second frame image of the second partial area is stored in the element of the matrix (i, j)=(1, 2). Here, DP (Dynamic Programming) matching is performed using (1, 1) as a leading end 510 and (M, N) as a terminal end 520. Then, the total of values (the similarity of the observation region) stored in the respective elements of the matrix from the leading end 510 to the terminal end 520 is regarded as a cost, and a route (combination) making the cost optimum is searched. In FIG. 5A, the elements selected as the optimum route by the DP matching are shown in gray. The search of the optimum route is performed wider a restraint that the frame images advance by 0 or 1 in an i-axis direction and 0 or 1 in a j-axis direction front the leading end 510 to the terminal end 520. Thus, it is possible to prevent a time series from being reversed in the route from the leading end 510 to the terminal end 520.

Next, some of the respective fiat e numbers selected as the optimum route when the frame images advance in a time series as described above are acquired as a first timing and a second timing. For example, the first timing and the second timing corresponding to the first timing are acquired at regular intervals on the basis of frame numbers of the moving image of the first partial area.

First, the reconstruction timing acquisition unit 214 sets a timing for thinning the moving image of the first partial area at regular intervals as the first timing. The interval may be set on the basis of the frame rate of the moving image of the first partial area and the frame rate of a combined moving image. For example, when the frame rate of the moving image of the first partial area is 10 frames/second and the frame rate of the combined moving image is 5 frames/second, the first timing is only required to be set at a 2-frame interval. Similarly, when the frame rate of the moving image of the first partial area is 12 frames/second and the frame rate of the combined moving image is 4 frames/second, the first timing is only required to be set at a 3-frame interval. That is, when the frame rate of the moving image of the first partial area is a frames/second and the frame rate of the combined moving image is b frames/second, the first timing is only required to be set at a (a/b)-frame interval. Further, when the moving image of the first partial area is acquired at a 2-frame interval, odd-number frames (i=1, 3, 5, . . . ) or even-number frames (i=2, 4, 6, . . . ) may be set as the first timing. The same applies to a case in which the moving image of the first partial area is acquired at an n-frame interval, and n combinations each starting at i=1, i=2, . . . , i=n−1 may be taken.

Here, a case in which the moving image of the first partial area is acquired at a 2-frame interval and odd-number frames (i=1, 3, 5 . . . ) are selected as the first timing will be considered as an example of describing processing to acquire the second timing. At this time, the reconstruction timing acquisition unit 214 acquires, for each of the frames (i=1, 3, 5, . . . ) of the first timing, a frame j corresponding to a frame i on the optimum route selected in the above processing as the second timing (that is, makes the frames i and j correspond to each other). However, when a plurality of frames j exist on the optimum route with respect to one frame i, frames j at which the similarity of the observation region becomes optimum are acquired as the second timing.

For example, when two frames (1, 1) and (1, 2) are selected as an optimum route where i=1 as shown in FIG. 5A, only a frame j having higher similarity (j=2 in the example of FIG. 5B) in the observation region is acquired. FIG. 5B shows an example of acquiring the first timing and the second timing on the basis of a case in which the frames i are odd-number frames. In FIG. 5B, the first timing corresponds to the frames {1, 3, 5, and 7}, and the second timing at frames {2, 4, 5, and 8} corresponding to the first timing is acquired. That is, the frames (1, 2), (3, 4), (5, 5), and (7, 8) of the two moving images are made to correspond to each other.

Similarly, the reconstruction timing acquisition unit 214 may acquire, for each of even-number frames (i=2, 4, 6, . . . ), a frame j at which the similarity of the observation region becomes optimum on an optimum route, and regard the frames i as the first timing and the frames j as the second timing. Alternatively, the reconstruction timing acquisition unit 214 may similarly calculate frames j corresponding to frames i with respect to the variation (a case in which the frames i are odd-number frames and a case in which the frames i are even-number frames when n=2) of the first timing, and select a combination with which the total of the similarity of the observation region becomes optimum to acquire the first timing and the second timing. Further, a method for selecting the combination is not limited to a method based on the total of the similarity, but may be, for example, a standard such as selecting the intervals of the second timing that are closer to regular intervals or a standard considering both these standards.

In addition, the first timing and the second timing may be acquired on the basis of the frame numbers of the moving image of the second partial area instead of the frame numbers of the moving image of the first partial area. In this case, the second timing may be acquired at the n-frame interval described above, and the first timing corresponding to the second timing may be calculated using an optimum route in the same manner as the above. Further, an optimum combination may be found by each of a pattern based on the moving image of the first partial area and a pattern based on the moving image of the second partial area. In addition, an optimum combination (for example, a combination with which the total of the similarity of the observation region becomes optimum) may be selected from both the patterns.

Note that even when the dynamic state of the observation region during capturing is not the same between the first projection data and the second projection data, thinning is performed on the basis of one of the first projection data and the second projection data, whereby it is possible to generate a combined moving image on a time base based on one of the first projection data and the second projection data. For example, when the change speed of the dynamic state of the observation region during capturing is different between the first projection data and the second projection data, it is possible to generate a combined moving image on the basis of a time base of one of the first projection data and the second projection data according to the speed. For example, when thinning is performed on the basis of one of the first projection data and the second projection data in which the change speed of the dynamic state of the observation region is faster, it is possible to remove frames having small movement information. Therefore, a reduction in the redundancy of data is allowed. On the other hand, when thinning is performed on the basis of one of the first projection data and the second projection data in which the speed of the observation region is slower, it is possible to generate a combined moving image without lacking information on detailed movement.

Further, thinning is not necessarily performed on the basis of one of the first projection data and the second projection data but may be performed on the basis of both the first projection data and the second projection data. For example, in a phase (a maximum respiration phase or a maximum expiration phase) in which information on the movement of the observation region is relatively small, thinning may be performed on the basis of projection data in which the movement of the observation region is faster. In other phases, thinning may be performed on the basis of projection data in which the movement of the observation region is slower. Thus, it is possible to generate a combined movement image maintaining information on detailed movement while reducing the redundancy of data.

Further, a thinning interval may be arbitrarily set by a user. For example, the user is allowed to set the frame rate of a combined movement image to be output and determine the thinning interval on the basis of the frame rate. Thus, it is possible to efficiently perform reconstruction only at a timing sufficient for generating a combined moving image in the second reconstruction processing unit 215 that will be described later.

Note that (1, 1) is the leading end 510 and (M, N) is the terminal end 520 in FIG. 5A but, the configuration is not limited to this, and DP matching in which a leading end and a terminal end are free may be performed. In this case, the leading end may be any frame at i=1 (left end column) or j=1 (upper end row), and the terminal end may be any frame at i=M (right end column) or j=N (lower end row). Further, the leading end and the terminal end may not be necessarily the end row and the end column of the matrix 500, respectively. If the acquisition of even-number frames i and frames j at which the similarity of the observation region becomes optimum is fixed in advance when thinning is performed at a 2-frame interval on the basis of the first projection data, it is possible to reduce a calculation amount by setting the leading end at any frame of i=2.

Further, the search of the route with which the total of the similarity becomes optimum is performed, but other statistical values including an average, a median, or the like other than the total may be used. According to the method, it is possible to search for an optimum route without relying on the distance of the route. Further, the search of a route is performed under the restraint that the frame images advance by 0 or 1 in the i-axis direction and 0 or 1 in the j-axis direction. However, a thinning axis direction and a thinning interval may be fixed in advance, and the frame images may be restricted to advance at the interval in the direction.

Further, when the thinning axis direction and the thinning interval are fixed in advance, frames that are not selected by thinning may be removed from the matrix 500 in advance. For example, when thinning is performed at a 2-frame interval in the i-axis direction, the number of rows in the i-axis direction of the matrix is set at M/2, whereby a reduction in the size of the matrix is allowed. Therefore, it is possible to reduce a calculation amount.

Further, a penalty may be provided depending on an advancing direction. For example, when the speed of the cyclic dynamic state of the observation region is close as the corresponding relationship of the dynamic state of a subject between respective capturing of first projection data and second projection data, the movement amount of an observation region is close between respective frames of the moving image of the first partial area and respective frames of the moving image of the second partial area. At this time, a route in which frame images advance one by one in both the i-axis direction and the j-axis direction becomes an optimum route in many cases. For this reason, when it is assumed that the change speed of the cyclic dynamic state of the observation region is close between respective capturing of the first projection data and the second projection data, the penalty provided when frame images advance by 1 in both the i-axis and the j-axis directions may be reduced (for example, the penalty=0). In addition, the penalty may be increased in other cases. According to this method, it is possible to robustly perform a route search even when an error occurs in the similarity of the observation region acquired in step S330.

Further, the search of the optimum route (the optimization of a combination of frame numbers) is performed in the two-dimensional matrix 500 in FIG. 5. However, when the similarity of the observation region in a plurality of translational movement amounts is acquired in step S330, the search of the optimum route may be performed using a three-dimensional matrix to which the axis of the translational movement amounts is added. In this case, it is possible to simultaneously perform the correspondence of the frame numbers and the optimization of the translational movement amounts between the frames. Further, like providing a penalty according to an advancing direction on a two-dimensional matrix, a penalty may be provided even in the advancing direction in the axis direction of the translational movement amounts in the case of a three-dimensional matrix.

As described above, the priority of a combination of first time positions and second nine positions is set according to the corresponding relationship of the dynamic state of the subject or the relative positions of the frame images between the first projection data and the second projection data. Then, on the basis of a combination haying higher priority among combinations having higher similarity of the observation region, the first time positions and the second time positions are regarded as the first timing and the second timing, respectively. Thus, it is possible to optimize a combination of the first timing and the second timing.

Note that an example of a DP algorithm is used as a method for optimizing a combination of the first timing and the second timing. However, other DP algorithms may be used. Further, a method using a probability model such as a hidden Markov model or other known methods may be used.

<S350: Reconstruction of Moving Images of Divided Areas> In step S350, the second reconstruction processing unit 215 acquires, from the first projection data, a moving image of a first divided area in which the entire capturing range of the first projection data is reconstructed at the first timing acquired in step S340. Similarly, the second reconstruction processing unit 215 acquires, from the second projection data, a moving image of a second divided area in which the entire capturing range of the second projection data is reconstructed at the second timing acquired in step S340. Then, the second reconstruction processing unit 215 outputs the acquired moving images of the first and second divided areas to the combined moving-image acquisition unit 216.

<S360: Acquisition of Combined Moving Image> In step S360, the combined moving-image acquisition unit 216 acquires a combined moving image in which respective corresponding frame images of the moving images of the first and second divided areas acquired in step S350 are combined together. Then, the combined moving-image acquisition unit 216 outputs the acquired combined moving image to the storage unit 203.

Here, the combined moving-image acquisition unit 216 combines the corresponding frame images of the first and second divided areas together. At this time, the combined moving-image acquisition unit 216 combines, on the basis of translational movement amount at which the similarity of the observation region acquired in step S330 becomes the highest, the frame images together so that corresponding positions between the frame images are matched to each other to generate a combined image. Alternatively, the combined moving-image acquisition unit 216 may align the corresponding frame images of the moving images of the first and second divided areas with each other in the present step and combine the frame images together so that the corresponding positions between the frame images are matched to each other on the basis of an alignment result to generate a combined image. Note that in the combined image, the pixel value of the pixels of an area in which two frame images are overlapped with each other may be a pixel value of any one of the frame images or may be an average of the pixel values of both the frame images. Alternatively, when the observation region is a lung, a weighted average may be used in which the weight of the pixel values of the frame images of the first divided area is increased in pixels on the side of the apex of the lung and in which the weight of the pixel values of the frame images of the second divided area is increased in pixels on the side of the base of the lung. In step S350, on the basis of a translational movement amount at which the similarity of the observation region acquired in step S330 becomes the highest, combined projection data may be reconstructed in which the first timing of the first projection data and the second timing of the second projection data, corresponding to the first timing of the first projection data, are combined together in a body axis direction. Thus, it is possible to reconstruct a combined moving image. In this case, the processing of step S360 becomes unnecessary.

<S370: Display of Combined Moving Image> In step S370, the display control unit 201 displays the combined moving image acquired in step S360 on the display unit 202.

For example, the display control unit 201 may MPR-display the respective frame images of the combined moving image or may display a cross section with an arbitrary angle. The display control unit 201 may receive an operation by the user via a GUI displayed on the display unit 202 and display the combined moving image changing the operation of reproducing or stopping the combined moving image, the switching or enlargement ratio of a cross section, and the direction of a cross section.

Note that the display control unit 201 may not necessarily display the combined moving image. Instead, the display control unit 201 may be configured to store or output the combined moving image generated in step S360 in or to the storage unit 203 or an external server. Thus, it is possible to obtain the same effect through the display of the stored combined moving image on a medical image viewer not shown.

In the manner described above, the processing of the information processing apparatus is performed. In the present embodiment, it is possible to generate a combined moving image in which respective frame images having phases of an observation region that correspond to each other between a plurality of moving images are combined together from a three-dimensional moving image obtained by dividing an area into pieces so as to include the entire area of the observation region and separately capturing the divided areas a plurality of times.

Next, a modified example of the first embodiment will be described below. Note that in the following description, the same constituting elements and processing as those described above will be denoted by the same symbols and their detailed descriptions will be omitted.

### MODIFIED EXAMPLE 1-1

In step S360, the positions of the frame images of the first and second divided areas are corrected to combine the frame images together so that the corresponding positions of the observation region are matched to each other between the frame images of the first and second divided areas. However, the configuration is not limited to this and in, for example, step S350, a moving image of the second divided area of which the position is corrected may be generated on the basis of the translational movement amount at which the similarity of the observation region acquired in step S330 becomes the highest. In this case, the frame images of the first and second divided areas are combined together without correcting the positions in step S360 to generate a combined moving image.

Further, without calculating a correction amount by the translational movement in step S330, the frame images of the first and second divided areas may be combined together on the basis of information on a capturing range (without correcting the translational movement) to generate a combined moving image. Thus, it is possible to reduce a calculation amount.

### MODIFIED EXAMPLE 1-2

In step S340, only the frame j having higher similarity of the observation region is acquired when the two frames (1, 1) and (1, 2) are selected as the optimum route at i=1 as shown in FIG. 5A. However, the configuration is not limited to this, and, for example, when a part of the combination selected as the optimum route includes the frames (1, 1), (1, 2), and (2, 3) as shown in FIG. 5A, a timing between i=1 and i=2 may be acquired as a candidate for the first timing by setting (1, 2) as (1.5, 2). As the timing between i=1 and i=2, it is possible to acquire, for example, i=1.5 that is the middle point between 1 and 2. At this time, the frame image of the first divided area corresponding to the time may be acquired in step S350.

Further, in the example of FIG. 5A, an integer j having optimum similarity to i=5 are selected from among j=4, 5, 6, and 7 in the embodiment when the frame j corresponding to i=5 is selected from the optimum route. In addition, values (that is, decimal numbers) obtained by dividing these values into sub-frames may be selected. The values may be acquired by, for example, calculating a function C(j) of a frame j that approximates each similarity to i=5 (applying the function to the similarity) and calculating the position (most analogous position) at which the function shows a maximum value. At this time, the frame image of the second divided area corresponding to the time may be acquired in step S350. Thus, it is possible to acquire a timing for reconstructing a divided area with higher time resolution than a moving image of a partial area.

### MODIFIED EXAMPLE 1-3

The reconstruction processing of the moving images of the partial areas in step S320 and the reconstruction processing of the moving images of the divided areas in step S350 may be performed under different reconstruction conditions. For example, in step S320, the moving images may be reconstructed with low resolution in order to reduce a calculation amount in step S320, and the moving images may be reconstructed with high resolution in step S350. Thus, on the basis of the first timing, the image of the first divided area is reconstructed from the first projection data under a first reconstruction condition at least partially different from a reconstruction condition under which the moving image of the first partial area is reconstructed. Further, on the basis of the second timing, the image of the second divided area is reconstructed from the second projection data under a second reconstruction condition at least partially different from a reconstruction condition under which the moving image of the second partial area is reconstructed.

Further, the moving images may be reconstructed by a reconstruction function (for example, a mediastinal condition) that suppresses a high-frequency noise component in step S320, and the user may reconstruct the moving image by other reconstruction functions to be observed in step S350. Thus, when the positions between the frame images are corrected by translational movement or the like in step S330, it is possible to perform robust processing by which influence by noise or the like is suppressed.

Further, the moving images may be reconstructed by a reconstruction function (for example, a lung field condition) that emphasizes a high-frequency component in order to correct the positions using fine structures inside the lung in step S320, and the moving images may be reconstructed by a different reconstruction function (for example, a mediastinal condition) in step S350. Alternatively, the moving images of the first and second divided areas may be reconstructed and acquired under a plurality of reconstruction conditions in step S350, and the moving images acquired under the respective reconstruction conditions may be combined together to generate a plurality of combined moving images in step S360. Then, selection by the user may be received via the operation unit 204, and a combined moving image under a reconstruction condition selected by the user may be displayed on the display unit 202 in step S370.

### MODIFIED EXAMPLE 1-4

A series of the processing to reconstruct the moving image of the first divided area in step S350 on the basis of the first timing acquired in step S340 is described above. However, the configuration is not limited to this, and, for example, the first timing acquired in step S340 and the first projection data may be stored in the storage unit 203 so as to correspond to each other, and the processing may proceed to step S350 when instructions to proceed the processing to step S350 are received from the user via the operation unit 204. The same processing may be performed also on the second timing and the second projection data.

Further, the user may specify, via the operation unit 204, the above at least one reconstruction condition for the moving images of the first and second divided areas in step S350. Then, in step S350, the second reconstruction processing unit 215 reconstructs the moving images of the first and second divided areas on the basis of the reconstruction condition specified by the user. In this case, as the minimum configuration of the information processing apparatus, the processing to step S340 and processing to store the resulting first and second timings so as to correspond to the first and second projection data, respectively, are only required to be performed. Then, processing to acquire these data and reconstruct and combine the projection data at the respective timings may be performed by other apparatuses.

### MODIFIED EXAMPLE 1-5

When the reconstruction condition in step S350 is the same as the reconstruction condition in step S320, an area other than the areas reconstructed in step S320 may be reconstructed and images after the reconstruction may be combined together to acquire the moving image of the divided areas. For example, when the range 430 shown in FIG. 4 is the first patrial area, in step S350 respective frame images are acquired in which only an area obtained by excluding the range 430 from the range 410 is reconstructed. Then, the respective frame images may be combined with the respective frame images of the first partial area to acquire the frame images of the first divided area. Thus, the restriction of a range reconstructed in step S350 is allowed. Therefore, it is possible to reduce a calculation amount relating to the reconstruction.

### Second Embodiment

In the first embodiment, moving images of first and second partial areas are acquired (reconstructed), and the phases of an observation region are made to correspond to each other. On the other hand, moving images of partial areas are not acquired (reconstructed) in the present embodiment, and the phases within a cycle of an observation region showing a cyclic dynamic state between the projection data of first and second partial areas are made to correspond to each other to generate a combined moving image.

Note that an observation region in the following embodiment is a lung like the first embodiment, and a description will be given using first projection data and second projection data as data captured by an X-ray CT apparatus. However, an observation region and a capturing apparatus are not limited to the lung and the X-ray CT apparatus.

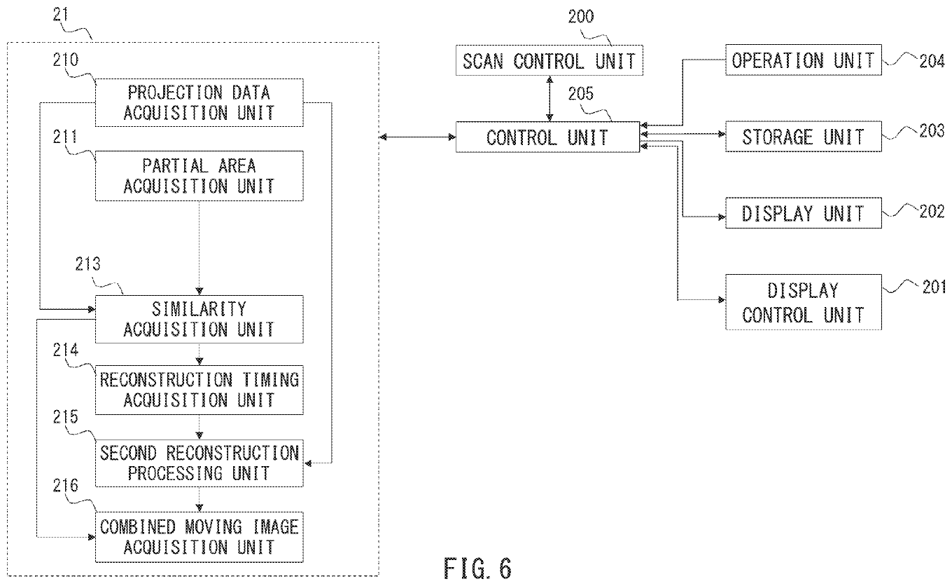

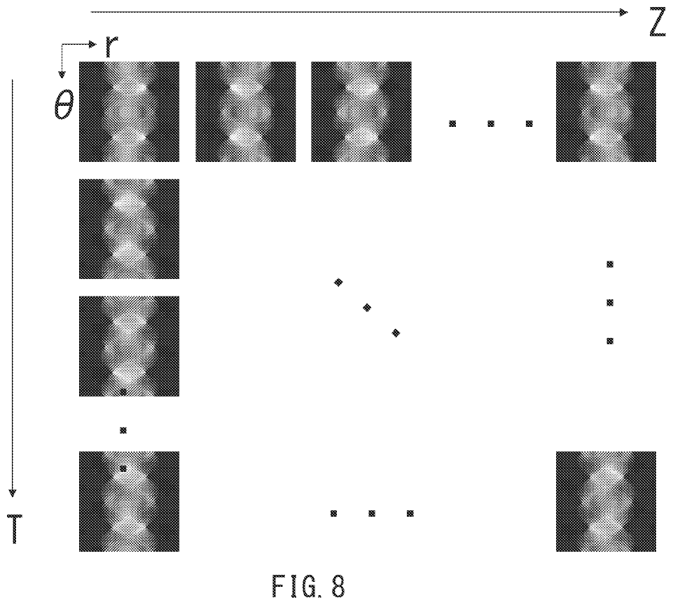

Hereinafter, the configuration and processing of the present embodiment will be described using FIGS. 6 and 8. FIG. 6 shows the configuration of an image processing apparatus according to the present embodiment. In the image forming apparatus according to the present embodiment, a first reconstruction processing unit 212 does not exist unlike the first embodiment. Further, in the present embodiment, processing performed by a similarity acquisition unit 213 is different from that performed in the first embodiment. Therefore, the function of the similarity acquisition unit 213 will be described. Other configurations have the same functions as those of the first embodiment. Therefore, respective constituting elements will be denoted by the same symbols, and their detailed descriptions will be omitted.

In the first embodiment, the similarity acquisition unit 213 acquires the similarity of an observation region using moving images of first and second partial areas reconstructed by the first reconstruction processing unit 212. On the other hand, the similarity acquisition unit 213 in the present embodiment compares first projection data with second projection data to acquire the similarity of an observation region. In the present embodiment, the similarity of an observation region is information that specifies projection data of a second partial area having a close phase within a cycle of the observation region showing a cyclic dynamic state in time-series projection data of a first partial area.

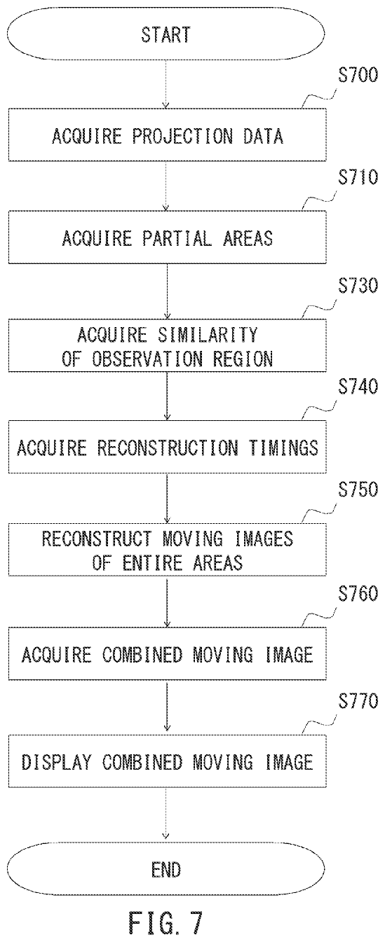

FIG. 7 shows the flowchart of an entire processing procedure performed by an information processing apparatus (console apparatus 20) according to the present embodiment. Since the processing of step S700, step S710, and steps S740 to S770 of the present embodiment is the same as that of step S300, step S310, and steps S340 to S370 of the first embodiment, its description will be omitted. Hereinafter, only a portion different from that of the flowchart shown in FIG. 3 will be described.

<S730: Acquisition of Similarity of Observation Region> In step S730, the similarity acquisition unit 213 acquires the similarity of an observation region between first projection data and second projection data that are acquired in step S700. Then, the similarity acquisition unit 213 outputs the acquired similarity of the observation region to a reconstruction timing acquisition unit 214 and a combined moving-image acquisition unit 216.

In the first embodiment, the similarity acquisition unit 213 compares a moving image of a first partial area with a moving image of a second partial area to acquire the similarity of an observation region. In the present embodiment, the similarity acquisition unit 213 compares projection data. corresponding to a first partial area in first projection data with projection data corresponding to a second partial area in second projection data to acquire the similarity of an observation region. That is, like the step S330 of the first embodiment, in step S730 the similarity acquisition unit 213 may move and compare the relative positions of the projection data of the first partial area and the projection data of the second partial area with each other to calculate the similarity.

Here, the projection data corresponding to the first partial area corresponds to the sinogram (sinogram of the first partial area) used to reconstruct the respective frame images of the first partial area in the first embodiment. Similarly, the projection data corresponding to the second partial area corresponds to the sinogram (sinogram of the second partial area) used to reconstruct the respective frame images of the second partial area in the first embodiment. That is, the sinogram of the first partial area is a sinogram generated from detection data collected from a position including the first partial area in first detection data.

Similarly, the sinogram of the second partial area is a sinogram generated from detection data collected from a position including the second partial area in second detection data. Note that the sinogram of the first partial area and the sinogram of the second partial area are time-series image data generated from detection data obtained by collecting the dynamic state of the observation region along a time series.

Accordingly, as shown in FIG. 8, a value where an arbitrary space (horizontal axis), an angle (vertical axis), a position in a body-axis direction, and a time in the sinogram of the first partial area are r, θ, z, and t1, respectively, is represented as S1 (r, θ, z, t1). Similarly, a value where an arbitrary space (horizontal axis), an angle (vertical axis), a position in a body-axis direction, and a time in the sinogram of the second partial area are r, θ, z, and t2, respectively, is represented as S2 (r, θ, z, t2). The space (horizontal axis), the angle (vertical axis), and the position in the body -axis direction show coordinates common between the sinogram of the first partial area and the sinogram of the second partial area that are controlled by the scan control unit 200.

In the present embodiment, the similarity of the observation region is acquired by comparing values at corresponding coordinates (r, θ, z) in the respective sinograms with each other in the sinogram of the first partial area and the sinogram of the second partial area at respective times. This processing is equivalent to processing in which the sinogram of the first partial area and the sinogram of the second partial area at the respective times are regarded as a three-dimensional image using (r, θ, z) as coordinate axes to calculate image similarity. Further, the frame number i of the moving image of the first partial area corresponds to t1, and the frame number j of the moving image of the second partial area corresponds to t2 in step S330 of the first embodiment.

Accordingly, the sinogram of the first partial area and the sinogram of the second partial area may be regarded as the moving image of the first partial area and the moving image of the second partial area of the first embodiment, respectively, and processing equivalent to the processing of step S330 may be performed to acquire the similarity of the observation region. However, the values of the sinograms are numerical values showing the decay of X-rays (integrated value in an irradiation direction). Therefore, it is difficult to acquire position information on the inner structure of the observation region in an irradiation direction (XY axes of a platform apparatus 10) without performing reconstruction processing. Therefore, it is difficult to finely align the inner structure of the observation region. Accordingly, it is preferable to acquire only a correction amount in the body-axis direction (Z-axis of the platform apparatus 10). Alternatively, the correction amount of a position may not be acquired in the present embodiment (correction amount: 0).

In the manner described above, the processing of the information processing apparatus is performed. In the present embodiment, the similarity of an observation region between the projection data of areas in which capturing ranges are overlapped with each other among a plurality of projection data captured by dividing the observation region is acquired, and a timing for reconstructing the entire capturing range is acquired. Then, a combined moving image in which a plurality of moving images obtained by reconstructing the entire capturing image are combined together is generated. In the present embodiment, moving images of partial areas are not acquired compared with the first embodiment. Therefore, it is possible to further reduce a calculation amount and a memory use amount for reconstruction.

The embodiments of the present disclosure are described above. The technology of the present disclosure is not limited to the above embodiments, but various modifications and/or deformations are possible within the technical scope of claims.

### Other Embodiments

Embodiment(s) of the present invention can also be realized by a computer of a system or apparatus that reads out and executes computer executable instructions (e.g., one or more programs recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

According to the present disclosure, it is possible to match the phases of the dynamic state of an observation region to each other with high accuracy between a plurality of moving images while suppressing a calculation amount for reconstruction.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2021-148065, filed on Sep. 10, 2021 which is hereby incorporated by reference herein in its entirety. In addition, this application claims the benefit of Japanese Patent Application No. 2021-148108, filed on Sep. 10, 2021 which is hereby incorporated by reference herein in its entirety.

What is claimed is:

1. An information processing apparatus comprising:
at least one memory storing a program; and
at least one processor which, by executing the program, causes the information processing apparatus to
acquire projection data obtained by dividing a subject into a first divided area and a second divided area and capturing the first divided area and the second divided area, the projection data including first projection data obtained by capturing a dynamic state of the subject in a first capturing range including the first divided area and second projection data obtained by capturing the dynamic state of the subject in a second capturing range including the second divided area,
acquire similarity relating to the dynamic state of the subject between the first projection data and the second projection data on a basis of projection data of a first partial area that is at least a part of the first capturing range in the first projection data and projection data of a second partial area that is at least a part of the second capturing range in the second projection data, and
acquire a first timing for reconstructing an image of the first divided area from the first projection data and a. second timing for reconstructing an image of the second divided area from the second projection data, on a basis of the similarity.

2. The information processing apparatus according to claim 1, wherein
each of the first partial area and the second partial area includes a part of an overlap area in which the first capturing range and the second capturing range overlap each other.

3. The information processing apparatus according to claim 1, wherein
the at least one processor which, by executing the program, further causes the information processing apparatus to acquire a combined moving image composed of a plurality of combined images in which a plurality of images of the first divided area and a plurality of images of the second divided area are combined together.

4. The information processing apparatus according to claim 1, wherein
the at least one processor which, by executing the program, further causes the information processing apparatus to acquire combined projection data in which the first projection data and the second projection data are combined together on a basis of the first timing and the second timing and reconstruct the combined projection data, thereby acquiring a combined moving image.

5. The information processing apparatus according to claim 1, wherein
the at least one processor which, by executing the program, further causes the information processing apparatus to acquire as the second timing a timing at which similarity of a phase in the dynamic state of the subject is high with respect to the first timing.

6. The information processing apparatus according to claim 1, wherein
the dynamic state of the subject is respiratory movement of the subject.

7. The information processing apparatus according to claim 6, wherein
the similarity is proximity of a phase within a respiration cycle in the respiratory movement.

8. The information processing apparatus according to claim 1, wherein
the at least one processor which, by executing the program, further causes the information processing apparatus to
move a relative position of the projection data of the first partial area or the projection data of the second partial area and compare the projection data of the first partial area with the projection data of the second partial area, thereby calculating the similarity, and
regard, on a basis of a combination having higher similarity from among combinations of first time positions in a time rage in which the first projection data is captured and second time positions in a time range in which the second projection data is captured, the first time positions as the first timing and the second time positions as the second timing.

9. The information processing apparatus according to claim 8, wherein
the at least one processor which, by executing the program, further causes the information processing apparatus to
set priority of the combinations of the first time positions and the second time positions according to a corresponding relationship of the dynamic state of the subject between the first projection data and the second projection data, and regard, on a basis of a combination having higher priority from among the combinations having the higher similarity, the first time positions as the first timing and the second time positions as the second timing.

10. The information processing apparatus according to claim 9, wherein
the at least one processor which, by executing the program, further causes the information processing apparatus to set priority of the combinations of the first time positions and the second time positions according to a movement amount of the relative position and regard the first time positions as the first timing and the second time positions as the second timing on a basis of the combination having the higher priority from among the combinations having the higher similarity.

11. The information processing apparatus according to claim 1, wherein
the projection data of the first partial area and the projection data of the second partial area are sinograms, and
the at least one processor which, by executing the program, further causes the information processing apparatus to compare values at corresponding coordinates in the respective sinograms in the projection data of the first partial area and the projection data of the second partial area with each other, thereby calculating the similarity.

12. An information processing apparatus comprising:
at least one memory storing a program; and
at least one processor which, by executing the program, causes the information processing apparatus to
acquire projection data obtained by dividing a subject into a first divided area and a second divided area and capturing the first divided area and the second divided area, the projection data including first projection data obtained by capturing a dynamic state of the subject in a first capturing range including the first divided area and second projection data obtained by capturing the dynamic state of the subject in a second capturing range including the second divided area,
acquire a moving image of a first partial area obtained by reconstructing an image of a first partial area that is a part of the first capturing range from the first projection data and a moving image of a second partial area obtained by reconstructing an image of the second partial area that is a part of the second capturing range from the second projection data,
acquire similarity in the dynamic state of the subject between respective frames of the moving image of the first partial area and the moving image of the second partial area, and
acquire a first timing for reconstructing a image of the first divided area from the first projection data and a second timing for reconstructing an image of the second divided area from the second projection data, on a basis of the similarity.

13. The information processing apparatus according to claim 12, wherein
each of the first partial area and the second partial area includes a part of an overlap area in which the first capturing range and the second capturing range overlap each other.

14. The information processing apparatus according to claim 12, wherein
the at least one processor which, by executing the program, further causes the information processing apparatus to acquire the image of the first divided area, which is reconstructed under a first reconstruction condition at least partially different from a reconstruction condition for reconstructing the moving image of the first partial area, from the first projection data on a basis of the first timing and an image of the second divided area, which is reconstructed under a second reconstruction condition at least partially different from a reconstruction condition for reconstructing the moving image of the second partial area, from the second projection data on a basis of the second timing.

15. The information processing apparatus according to claim 14, wherein
the first divided area is different in at least a partial area from the first partial area, the first reconstruction condition is a reconstruction condition in which the first divided area is a reconstruction range, the second divided area is different in at least a. partial area from the second partial area, and the second reconstruction condition is a reconstruction condition in which the second divided area is a reconstruction range.

16. The information processing apparatus according to claim 14, wherein
the at least one processor which, by executing the program, further causes the information processing apparatus to acquire a combined moving image composed of a plurality of combined images in which a plurality of images of the first divided area and a plurality of images of the second divided area are combined together.

17. The information processing apparatus according to claim 12, wherein
the at least one processor which, by executing the program, further causes the information processing apparatus to acquire combined projection data in which the first projection data and the second projection data are combined together on a basis of the first timing and the second timing and reconstruct the combined projection data, thereby acquiring a combined moving image.

18. The information processing apparatus according to claim 12, wherein
the at least one processor which, by executing the program, further causes the information processing apparatus to acquire as the second timing a timing at which similarity of a phase in the dynamic state of the subject is high with respect to the first timing.

19. The information processing apparatus according to claim 12, wherein
the dynamic state of the subject is respiratory movement of the subject.

20. The information processing apparatus according to claim 19, wherein
the similarity is proximity of a phase within a respiration cycle in the respiratory movement.

21. The information processing apparatus according to claim 12, wherein
the at least one processor which, by executing the program, further causes the information processing apparatus to determine a frame rate of the reconstructed moving image of the first partial area according to an observation site of the subject included in the first capturing range.

22. The information processing apparatus according to claim 12, wherein
the at least one processor which, by executing the program, further causes the information processing apparatus to determine a frame rate of the reconstructed moving image of the first partial area according to a change in the dynamic state of the subject.

23. The information processing apparatus according to claim 12, wherein
a frame rate of the moving image of the first partial area is higher than a frame rate of a combined moving image of the image of the first divided area reconstructed at the first timing and the image of the second divided area reconstructed at the second timing.

24. The information processing apparatus according to claim 12, wherein
the at least one processor which, by executing the program, further causes the information processing apparatus to
move a relative position of a frame image of the moving image of the first partial area or the moving image of the second partial area, thereby calculating the similarity, and
regard, on a basis of combinations having the higher similarity from among combinations of first time positions in a time rage in which the first projection data is captured and second time positions in a time range in which the second projection data is captured, the first time positions as the first timing and the second time positions as the second timing.

25. The information processing apparatus according to claim 24, wherein
the at least one processor which, by executing the program, further causes the information processing apparatus to set priority of the combinations of the first time positions and the second time positions according to a corresponding relationship of the dynamic state of the subject between the first projection data and the second projection data, and regard, on a basis of a combination having the higher priority from among the combinations having the higher similarity, the first time positions as the first timing and the second time positions as the second timing.

26. The information processing apparatus according to claim 24, wherein
the at least one processor which, by executing the program, further causes the information processing apparatus to set priority of the combinations of the first time positions and the second time positions according to a movement amount of the relative position and regard the first time positions as the first timing and the second time positions as the second timing on a basis of the combination having the higher priority from among the combinations having the higher similarity.

27. An information processing method causing a computer to execute the steps of:
acquiring projection data obtained by dividing a subject into a first divided area and a second divided area and capturing the first divided area and the second divided area, the projection data including first projection data obtained by capturing a dynamic state of the subject in a first capturing range including the first divided area and second projection data obtained by capturing the dynamic state of the subject in a second capturing range including. the second divided area;
acquiring similarity relating to the dynamic state of the subject between the first projection data and the second projection data on a basis of projection data of a first partial area that is at least a part of the first capturing range in the first projection data and projection data of a second partial area that is at least a part of the second capturing range in the second projection data; and
acquiring a first timing for reconstructing an image of the first divided area from the first projection data and a second timing for reconstructing an image of the second divided area from the second projection data, on a basis of the similarity.

28. An information processing method causing a computer to execute the steps of:
acquiring projection data obtained by dividing a subject into a first divided area and a second divided area and capturing the first divided area and the second divided area, the projection data including first projection data obtained by capturing a dynamic state of the subject in a first capturing range including the first divided area and second projection data obtained by capturing the dynamic state of the subject in a second capturing range including the second divided area;
acquiring a moving image of a first partial area obtained by reconstructing a first partial area that is a part of the first capturing range from the first projection data and a moving image of a second partial area obtained by reconstructing a second partial area that is a part of the second capturing range from the second projection data;
acquiring similarity in the dynamic state of the subject between respective frames of the moving image of the first partial area and the moving image of the second partial area; and
acquiring a first timing for reconstructing an image of the first divided area from the first projection data and a second timing for reconstructing an image of the second divided area from the second projection data, on a basis of the similarity.

9. A non-transitory computer readable medium that stores a program, wherein the program causes a computer to execute the steps of:
acquiring projection data obtained by dividing a subject into a first divided area and a second divided area and capturing the first divided area and the second divided area, the projection data including first projection data obtained by capturing a dynamic state of the subject in a first capturing range including the first divided area and second projection data obtained by capturing the dynamic state of the subject in a second capturing range including the second divided area;
acquiring similarity relating to the dynamic state of the subject between the first projection data and the second projection data on a basis of projection data of a first partial area that is at least a part of the first capturing range in the first projection data and projection data of a second partial area that is at least a part of the second capturing range in the second projection data; and
acquiring a first timing for reconstructing an image of the first divided area from the first projection data and a second timing for reconstructing an image of the second divided area from the second projection data, on a basis of the similarity.

30. A non-transitory computer readable medium that stores a program, wherein the program causes a computer to execute the steps of:
acquiring projection data obtained by dividing a subject into a first divided area and a second divided area and capturing the first divided area and the second divided area, the projection data including first projection data obtained by capturing a dynamic state of the subject in a first capturing range including the first divided area and second projection data obtained by capturing the dynamic state of the subject in a second capturing range including the second divided area;
acquiring a moving image of a first partial area obtained by reconstructing a first partial area that is a part of the first capturing range from the first projection data and a moving image of a second partial area obtained by reconstructing a second partial area that is a part of the second capturing range from the second projection data;
acquiring similarly in the dynamic state of the subject between respective frames of the moving image of the first partial area and the moving image of the second partial area; and
acquiring a first timing for reconstructing an image of the first divided area from the first projection data and a second timing for reconstructing an image of the second divided area from the second projection data, on a basis of the similarity.
